(19) United States
(12) Patent Application Publication
(10) Pub. No.: US 2021/0322974 A1
(43) Pub. Date: Oct. 21, 2021

(54) MICROFLUIDIC DEVICES

(71) Applicant: HEWLETT-PACKARD DEVELOPMENT COMPANY, L.P., Spring, TX (US)

(72) Inventor: Alexander Govyadinov, Corvallis, OR (US)

(73) Assignee: HEWLETT-PACKARD DEVELOPMENT COMPANY, L.P., Spring, TX (US)

(21) Appl. No.: 17/268,339

(22) PCT Filed: Nov. 14, 2018
(86) PCT No.: PCT/US2018/060918

§ 371 Date: Feb. 12, 2021

(51) Int. Cl. B01L 3/00; B01L 7/00; C12Q 1/686

(52) U.S. Cl. CPC B01L 3/50273; B01L 7/525; B01L 3/502715; C12Q 1/686; B01L 2200/027; B01L 2400/0439; B01L 2300/0877; B01L 2300/0883; B01L 2300/1827; B01L 2300/1894; B01L 2400/0496; B01L 2300/088

(57) ABSTRACT

A microfluidic device including: a transport channel having an inlet and an outlet; a plurality of pump loops extending along the transport channel, wherein each of the plurality of pump loops includes: a first branch, a second branch, and a first connecting section connecting the first branch and the second branch, wherein the first branch includes a first opening and the second branch includes a second opening, and wherein the first opening and the second opening are in direct fluid communication with the transport channel; an actuator positioned in the first branch; and a heater positioned to heat fluid in a portion of the pump loop.

### BACKGROUND

Microfluidics applies across a variety of disciplines including engineering, physics, chemistry, microtechnology and biotechnology. Microfluidics involves the study of small volumes, e.g., microliters, picoliters, or nanoliters, of fluid and how to manipulate, control and use such small volumes of fluid in various microfluidic systems and devices such as microfluidic devices or chips. For example, microfluidic biochips (which may also be referred to as “lab-on-chip”) are used in the field of molecular biology to integrate assay operations for purposes such as analyzing enzymes and DNA, detecting biochemical toxins and pathogens, diagnosing diseases, etc.

Polymerase Chain Reaction (PCR) is a method for amplifying nucleic material. PCR consists of mixing a sample to be amplified with primers and nucleotides. The two strands of the double helix of the sample are physically separated using heat. This is generally referred to as “melting” or “denaturing.” Next, the temperature is lowered and the primers bind to the complementary sequences of DNA. This is known as “annealing”. The two DNA strands then become templates for DNA polymerase to enzymatically assemble new DNA strand from the nucleotides. This is known as “extension”. The cycle of these steps is repeated, with the number of copies of the original sample doubling each time.

### BRIEF DESCRIPTION OF THE DRAWINGS

Features of the present specification are illustrated by way of example and not limited in the following figure(s), in which like numerals indicate similar but not necessarily identical elements.

FIG. 1 shows an example of a microfluidic device consistent with the present specification.

FIG. 2 shows an example of a microfluidic device consistent with the present specification.

FIG. 3 shows an example of a microfluidic device having pump loops on both sides of the transport channel consistent with this specification.

FIG. 4 shows an example of a microfluidic device with a pump loop having a serpentine section consistent with this specification.

FIG. 5 shows a flowchart for a method of performing PCR consistent with the present specification.

FIG. 6 shows a microfluidic device with multiple transport channels consistent with this specification.

FIG. 7 shows a microfluidic device with multiple transport channels consistent with this specification.

FIG. 8 shows an array of microfluidic devices capable of parallel processing samples in an example consistent with this specification.

FIG. 9 shows an array of microfluidic devices capable of parallel processing samples in an example consistent with this specification.

FIG. 10 shows an array of microfluidic devices according to an example consistent with this specification.

FIG. 11 shows an example of a microfluidic device in an example consistent with this specification.

### DETAILED DESCRIPTION

This specification incorporates WO2018/017120 by the same inventor, published Jan. 25, 2018, and titled Microfluidic Devices in its entirety by reference. This specification also incorporates U.S. Pat. No. 9,724,920, issued Aug. 8, 2017, and titled Molded Die Slivers with Exposed Front and Back Surfaces and U.S. 2016/0001558 A1, filed Dec. 13, 2013, and titled Molded Printhead, in their entirety by reference. PCT/US2017/062935 filed Nov. 22, 2017 and PCT/US2017/063107 filed Nov. 22, 2017 are also incorporated by reference in their entirety.

For simplicity and illustrative purposes, the present specification is described by referring mainly to an example thereof. In the following description, numerous specific details are set forth in order to provide a thorough understanding of the present specification. It will be readily apparent, however, that the present specification may be practiced without limitation to these specific details. In other instances, some methods and structures have not been described in detail so as not to unnecessarily obscure the present specification. As used herein, the terms “a” and “an” are intended to denote at least one of a particular element, the term “includes” means includes but not limited to, the term “including” means including but not limited to, and the term “based on” means based at least in part on.

Additionally, it should be understood that the elements depicted in the accompanying figures may include additional components and that some of the components described in those figures may be removed and/or modified without departing from scopes of the elements disclosed herein. It should also be understood that the elements depicted in the figures may not be drawn to scale and thus, the elements may have different sizes and/or configurations other than as shown in the figures.

As used in this specification and the associated claims, the term “fluid” is used in the context and terminology of microfluidic devices. Accordingly, fluid includes liquid compositions which include additional phases, such as colloids, slurries, emulsions, etc. Fluid does not include, unless explicitly stated, gases without liquids (e.g., air) and/or plasmas (i.e., the state of matter of ionized particles).

Unless otherwise stated, all values include an implicit tolerance of 20% from the recited value. So a range of 100 to 200 C. would cover 80 to 240 C. because 20% of 100 is 20 so 100 covers 80 to 120 and 20% of 200 is 40 so 200 covers 160 to 240. When provided, alternate tolerances will include an appropriate +/− indicator (or +firstvalue/−secondvalue) to provide specificity.

Among other examples, this specification describes a microfluidic device including: a transport channel having an inlet and an outlet; a plurality of pump loops extending along the transport channel, wherein each of the plurality of pump loops includes: a first branch, a second branch, and a first connecting section connecting the first branch and the second branch, wherein the first branch includes a first opening and the second branch includes a second opening, and wherein the first opening and the second opening are in direct fluid communication with the transport channel; an actuator positioned in the first branch; and a heater positioned to heat fluid in a portion of the pump loop.

Among other examples, this specification also describes a method of performing Polymerase Chain Reaction (PCR) including: supplying a fluid comprising master mix to an inlet of a transport channel of a microfluidic device, said microfluidic device including a plurality of pump loops, each of the plurality of pump loops including: a first branch, a second branch, and a connecting section connecting the first branch and the second branch, wherein the first branch includes a first opening and the second branch includes a second opening, and wherein the first opening and the second opening are in direct fluid communication with the transport channel; a heater heating a portion of the pump loop; and an actuator positioned in the first branch; and activating the actuators in the plurality of pump loops to induce the fluid to be transported from the inlet, through the transport channel, and to an outlet of the transport channel such that the fluid passes through multiple heating zones in multiple loops.

This specification also describes a microfluidic device including: a first and second transport channel, each having an inlet and an outlet; a plurality of connecting passages connecting the first and second transport channels, wherein each of the plurality of the connecting channels includes: a first actuator near the first transport channel; and a second actuator near the second transport channel.

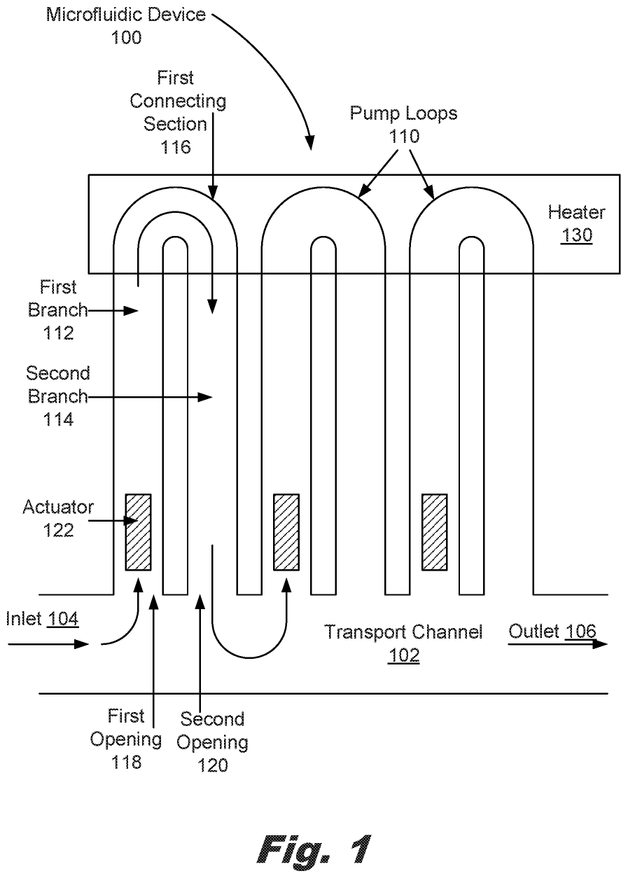

Turning now to the figures, FIG. 1 shows an example of a microfluidic device (100) consistent with the present specification. The microfluidic device (100) includes: a transport channel (102) having an inlet (104) and an outlet (106); a plurality of pump loops (110) extending along the transport channel (102), wherein each of the plurality of pump loops (110) includes: a first branch (112), a second branch (114), and a first connecting section (116) connecting the first branch (112) and the second branch (114), wherein the first branch (112) includes a first opening (118) and the second branch (114) includes a second opening (120), and wherein the first opening (118) and the second opening (120) are in direct fluid communication with the transport channel (102); an actuator (122) positioned in the first branch (112); and a heater (130) positioned to heat fluid in a portion of the pump loop (110).

The microfluidic device (100) includes multiple zones with different temperatures. The microfluidic device (100) pumps provided liquid from the inlet (104) to the outlet (106). In an example, the provided liquid includes master mix and a sample to be amplified. The heated zones are arranged to provide a time-temperature profile to perform the steps of PCR. In contrast with methods which heat the sample container and other components, in the present design the hot zones are static while the liquid flows through them. As a result, only the liquid needs to be brought to temperature, minimizing the heat needed to perform the temperature change. This allows quick heating and/or cooling of the liquid. This in turn allows rapid cycling.

The microfluidic device may be fabricated using a variety of approaches. In one example, the system is formed in silicon using masks/etching/deposition techniques. This approach allows integration of the actuator (122) and heater (130) elements into substrate. The downside with this approach is the relatively high cost of silicon and the potential for individual defects to require scrapping the whole device. While silicon oxide, silicon nitride (SiN), and/or silicon carbide (SiC) provide suitable chemical resistance, other options for the device (100) are also attractive. For example, plastics and glass can be used as substrates.

In an example, the device (100) is made from silicon components which have been overmolded to form the channels, loops, and other flow components of the device. The heater (130) and/or actuators (122) may still be provided on slivers of silicon. More discussion of the approach of integrated molding of silicon components into other structures may be found, for example, in U.S. Pat. No. 9,724,920, The body of the device (100) may be made from a molded polymer, e.g., polystyrene. The body of the device (100) may include thermoset polymer, e.g., epoxy. In an example, the device (100) is formed using a photoresist, either negative or positive. One exemplary material is SU-8, an epoxy. The device (100) may be a silicone or use a silicone mold. In an example, the device (100) includes a lower, molded component and an upper component. Molding, heat forming, casting, and any other suitable method of forming small scale channels may be used to form and/or assemble the device (100).

The transport channel (102) provides connection between the inlet (104) and the outlet (106). The transport channel (102) allows normalization of the pressure at the start of the pump loops (110). This allows each pump loop to function without requiring a pressure gradient of the sum of the pressure drops across all of the pump loops (110).

The inlet (104) provides fluid to the device (100) to be processed through the respective zones. The inlet (104) may be a port. The inlet (104) may be an outlet (106) from another device (100). The inlet (104) may be attached to a reservoir. The inlet (104) may have a higher pressure than the outlet (106).

The outlet (106) provides fluid from the device (100) which has passed through the time-temperature profile. The outlet (106) may be a port. The outlet (106) may supply another device (100).

The device (100) includes a plurality of pump loops (110) which extend from the transport channel (102). The pump loops (110) provide a local flow. This flow moves the fluid through the pump loop and out the second opening (120) on the second branch (114). Each pump loop (110) flows away from the transport channel (102) before turning and returning to the transport channel (102). The pump loops (110) may extend at a 90 degree angle from the transport channel (102). This may increase packing density. However, other angles will also allow useful geometries. For example, the transport channel (102) may be arranged as a circle with the pump loops (110) extending out like petals of a flower. Other geometries allow other configurations of heating zone(s) which may minimize the number of components required, the amount of electricity consumed, or other variables.

The pump loop (110) includes a first branch (112) which extends from the transport channel (102). Fluid flows from the transport channel (102) into the pump loop (110). The first branch contains the actuator (122) which provides the pumping in the pump loop (110). The first branch connects to the transport channel (102) through the first opening (118). The first branch (112) may be straight. The first branch (112) may include a curve. The first branch (112) may include a serpentine to increase the length of the first branch (112).

The second branch (114) is the return branch of the pump loop (110). The second branch brings the pumped fluid back to the transport channel where it can be provided to the next pump loop (110) in the series. In some examples, the second branch does not contain an actuator (122). However, the second branch (114) may also contain an actuator (122). Including an actuator (122) in the second branch (114) may provide additional control options, such as the ability to reverse flow in the associated pump loop (110). The second branch (114) connects to the transport channel (102) through the second opening (120).

The pump loop (110) includes a first connecting section (116) connecting the first branch (112) and the second branch (114). In FIG. 1, the connecting section (116) is also curved to form an elbow. The connecting section (116) may contain the heater (130) and/or may receive heat from the heater (130). The connecting sections (116) from multiple adjacent pump loops (110) may be heated by the same heater (130). The heater (130) may be remote to the pump loops (110) and heat based on conduction of heat through the material making up the device (100).

The first opening (118) and the second opening (120) are in direct fluid communication with the transport channel (102). The first opening (118) receives fluid from the transport channel (102). The second opening (120) returns fluid to the transport channel (102). These roles can be reversed using a second actuator (122), for example, in the second branch (114). A pump loop (110) of the plurality of pump loops (110) may a second actuator (122) in the second branch (114) of the pump loop (110). The second actuator (122) may be near the second opening (120). In an example, the first opening (118) has a smaller width and/or a smaller depth compared with other portions of the first branch (112). This narrowing of the first opening (118) may help contain a drive bubble formed by an actuator (122) from spilling back into the transport channel (102).

The pump loops (110) may have cross sectional areas of between about 10×5 micrometers² to about 200×500 micrometers². The cross sectional areas may vary outside of this range. The pump loops (110) may have diameters, widths, and/or depths that are between about 5 micrometers and about 500 micrometers. For example, the pump loops (110) may be 5 micrometers by 10 micrometers. The pump loops (110) may be 200 micrometers by 100 micrometers. In an example, the pump loops (110) are approximately 20 micrometers by 20 micrometers The cross sectional area of the transport channel (102) may be larger than the cross sectional areas of the pump loops (110). For instance, the cross sectional area of the transport channel (102) may be between about 200×50 micrometers² to about 500×100 micrometers². For instance, the transport channel (102) may be comparable with the depths of the pump loops (110) or significantly deeper than the pump loops (110).

The actuator (122), which is also referenced herein as a pump (e.g. of the pump loop), may be positioned in a respective first branch (112) of a pump loop (110). The actuator (122) may be located near the first opening (118). For instance, fluid from the transport channel (102) may be delivered into the first branches (112) through the first openings (118) and may flow over the actuators (122). The actuators (122) facilitate flow of the fluid through the respective pump loops (110) through application of pressure to the fluid. For instance, the actuators (122) may induce a traveling wave in the fluid to move the fluid through the pump loops (110). In an example, the actuators (122) are resistors that, when activated (e.g., by a thin film transistor), generate sufficient heat to vaporize fluid around the resistors, creating steam drive bubbles that push fluid through the pump loops (110).

In an example, the actuator (122) is a thermoresistive element which employs a thermal resistor formed on an oxide layer on a top surface of a substrate and a thin film stack applied on top of the oxide layer, in which the thin film stack includes a metal layer defining the thermoresistive element, conductive traces and a passivation layer.

In an example, the actuators (122) include piezoelectric elements, in which electrical current may selectively be applied to a piezoelectric member (by, for example, a field effect transistor) to deflect a diaphragm to push fluid through the pump loops (110). The actuators 122 may be other forms of presently available and/or future developed actuators such as electrostatic driven membranes, electro-hydrodynamic pulse pumps, magneto-strictive and other displacement devices.

Activation of the actuators (122) may cause fluid to be expelled from the pump loops (110) through the second opening (120). Activation of the actuators (122) may cause fluid to be drawn into the pump loops (110) through the first openings (118). Thus, by selectively activating the actuators (122), e.g., in a sequential arrangement, fluid that is initially received through the inlet (104) may be moved through the pump loops (110) to the outlet (106). The fluid may be conveyed by a wave through the pump loops (110). To ensure that the fluid flows through the pump loops (110) from the inlet (104) to the outlet (106) of the transport channel (102), the pump loops (110) may be enclosed except for the first opening (118) and the second opening (120).

The heater (130) is positioned to heat fluid in a portion of the pump loop (110). The heater (130) may heat fluid in one portion of the pump loop (110) while not heating fluid in other portions of the pump loop (110). For example, in FIG. 1, the heater (130) heats the fluid in the first connecting section (116) but need not directly heat fluid in the first branch (112) and/or second branch (114). The heater (130) may provide heating to multiple pump loops (110). In an example, these are multiple adjacent pump loops (110) as shown in FIG. 1.

The heater (130) may heat a single pump loop (110). In an example, the device (100) includes multiple individual heaters (130). Individual heaters (130) may allow each pump loop (110) to be heated to a different temperature. These individual heaters (130) may be implemented in a variety of methods. One approach is to use a resistive heater similar to the element used in the bubble forming mechanism of a thermal inkjet. This has the advantage of allowing the heaters (130) and the actuators (122) to be produced with the same processes. The heater (130) may be run at a lower duty cycle and/or current to perform the desired heating without forming a vapor bubble. In some examples, the heaters (130) and the actuators (122) are capable for performing both roles. For example, the actuator (122) may provide heating between firings. Similarly, a pair of resistive heaters (130) may be located in the first branch (112) and both using for pumping, both used for heating, and/or split between both operations.

In an example, the actuators (122) are used as heaters (130) during a pre operation phase to bring the device (100) up to a steady state operating temperature. Once that temperature is reached, heating may be left to dedicated heaters (130) and the actuators (122) used to move fluid through the device (100). This use of the actuators (122) may reduce the time to prepare the device (100) for use.

Each of the plurality of pump loops (110) may have a second heater (130) to apply heating to a separate portion of the pump loop (110). For example the first and second heaters (130) may be located in the first branch (112) and second branch (114) respectively. In an example, the first heater (130) heats the first connecting section (116) and a second heater (130) heats both the first branch (112) and the second branch (114). Placing individual heaters (130) at various points in the pump loop (110) allows control over the temperature profile of the fluid as it passes through the pump loop (110).

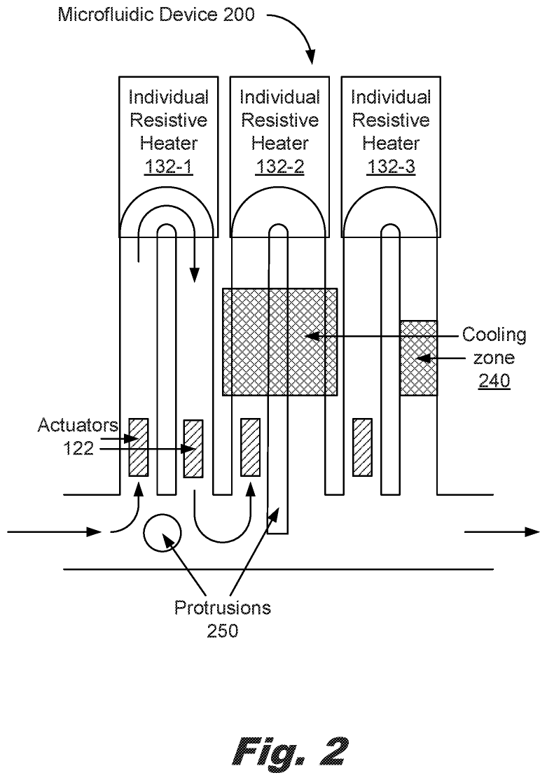

FIG. 2 shows an example of a microfluidic device (200) consistent with the present specification. The microfluidic device (200) of FIG. 2 adds additional features which may be mixed and matched in any combination to form useful devices (200). FIG. 2 shows individual resistive heaters (132-1 to 3), a second actuator (122) in a pump loop (110), cooling zones (240), and protrusions (250).

The inclusion of a second actuator (122) in a pump loop (110) may be used to provide additional pumping by coordinating with the first actuator (122). The second actuator (122) may be used to reverse the flow of fluid in the pump loop (110). The second actuator (122) may be in the second branch (114) of the pump loop (110). The second actuator (122) may be near the second opening (120) of the second branch (114) of the pump loop (110). The second actuator (122) may be located in the first branch (112) near the first actuator (122). The second actuator (122) may be located in the connecting section (116).

Individual resistive heaters (132) have been discussed above under heaters (130). Individual resistive heaters (132) provide additional control over the temperature profile of the fluid passing through the microfluidic device (200) compared with larger heaters (130). This additional control increases the number of control and/or power lines that need to be provided. This additional control may increase the complexity of other components of the microfluidic device (200).

Individual heaters (132) may be provided for different portions of a given pump loop (110). For example, a first individual heater (132-1) may be provided in the first branch (112) of a pump loop (110); a second individual heater (132-2) may be provided in the first connecting section (116) of the pump loop (110); and a third individual heater (132-3) may be provide in the second branch (114) of the pump loop (110). Other combinations are similarly possible, allowing the temperature-time profile of the fluid passing through the microfluidic device (200) to be arranged as desired to run a given reaction, e.g., PCR. For example, the combination of heater (130), individual heaters (132), and cooling zones (240) can be used to create the desired time-temperature profiles for denaturation (melting), annealing (attaching primers), and elongation (extension by polymerase) of DNA. For example, the denaturation step may be performed at 92 to 100 degrees C., and preferably at 94 to 98 degrees C. Denaturation may take approximately 20 to 30 seconds. In some examples, shorter times are preferred for example in a range of 1 to 5 seconds. The described designs with their low thermal mass allow potentially even shorter denaturation phases, for example, on the order of 1-10 milliseconds. Because the exposure time at the higher denaturation temperature may be kept short, higher temperatures may be used without the associated thermal degradation and other issues.

The annealing step may be performed at 48 to 72 degrees C. Annealing may be primer dependent; accordingly, having the ability to adjust the annealing temperature independent of the other zones of the microfluidic system allows flexibility to accommodate different primers. Annealing may take approximately 30 seconds. Annealing times may be reduced, for example to between 1 to 10 seconds. Under some conditions, Annealing times may be further reduced to 50 to 500 milliseconds. Because of the low thermal mass of the test sample, the temperature may be rapidly cycled compared with conventional techniques. This may greatly increase the speed and throughput compared with previously known PCR temperature-time profiles. Moving to higher throughput speeds is facilitated by higher primer concentrations and higher enzyme concentrations. For example, the primer concentration may be 10 to 50 times higher than conventional PCR concentrations. Similarly, enzyme concentrations may be 10 to 50 times higher than conventional PCR concentrations. Higher concentrations of enzymes and primers facilitate shorter cycle times by reducing diffusion times.

Extension may be performed at 68 to 80 degrees C. The extension temperature may vary depending on the particular polymerase used. Depending on the annealing temperature, the elongation temperature may overlap with the annealing temperature. Extension time depends on the number of bases to be copied, with 1000 bases a minute being a widely used rule of thumb. This allows rapid cycling of smaller lengths of nucleic material. The ability to loop a sample a number of times in a controlled temperature pump loop (110) may allow flexibility in tuning the extension time used. Similarly, the use of preheating and/or final extension before or after a set of cycles may be included by controlling the time-temperature profile of the microfluidic device (200).

In some examples, the PCR cycle is 10 seconds, 2 seconds, 1 second, 0.5, seconds, 0.1 seconds, and/or shorter times. The devices described herein support rapid thermal cycling by moving the liquid between zones without moving containment or other materials. The test volume has a small thermal load being moved through stable thermal zones, allowing rapid heating and/or cooling. Adjustments to the solution composition may facilitate faster cycling. See, for example, “Extreme PCR: Efficient and Specific DNA Amplification in 15-60 Seconds,” Clinical Chemistry, clinchem.2014.228304.

Similarly, the temperatures of the various regions may be changed over time. For example, in touchdown (or stepdown) PCR, the annealing temperature may be reduced in later cycles to increase specificity. A variety of PCR techniques may benefit from the ability to modify time, temperatures, chemical composition of the fluid, and/or other parameters over the cycles of the amplification. Similarly, hot start and/or cold finishes may be added and/or optionally added depending on the reagents used with the microfluidic system (200).

Cooling zones (240) facilitate rapid cycling of the temperature of the fluid passing through the device (200). Cooling as used herein may be cooling relative to ambient temperature. Cooling may also be relative to the temperature of the fluid but still warmer than the environment. For example, after melting a DNA sample, it may be desirable to cool the temperature to facilitate forming the new strands of DNA using polymerase. This cooling temperature may still be above room temperature.

Cooling zones may use a heat sink to cool the fluid. Depending on the temperature, the heat sink may be active or passive. Active cooling may include the use of a chiller or similar device to cool the heat sink. Passive cooling may depend on radiation, convection, and/or conductivity to cool the device. In some examples, the cooling zone is a metal which is either actively chilled or passive sinks heat away from the fluid. The metal surface may include a protective coating, for example, an oxide coating. However, some oxides are thermal insulators and may reduce the effectiveness of the metal as a heat sink. One advantage to a metal surface a heat sink is that such a surface can be formed with the same processes used to form the heater (130) and/or actuator (122). For example, the metal surface may be formed during a metallization step.

The cooling zone (240) may be organized into regions and/or subzones. In one example, a large cooling zone (240) is created and then its cooling is limited by local individual resistive heaters (132). This provides a way to create small cooling elements, namely shield part of the fluid from a large cooling element with a heater.

In an example, the cooling zone (240) is located downstream of the heater (130) or heating zone. In an example, a cooling zone is provided prior to the heating zone. Heating and/or cooling zones (240) may also be applied to the transport channel (102). In an example, the cooling zone includes the transport channel (102), the first opening (118), the second opening (120), a portion of the first branch (112), and a portion of the second branch (114).

The microfluidic device (200) may include protrusions (250). Protrusions (250) are positioned in the transport channel (102) to facilitate fluid flow into a first opening (118) of a pump loop (100). Protrusions (250) may be located in the transport channel (102) between a first opening (118) and a second opening (120). In this position, the protrusion (250) increases the resistance to flow in the transport channel and helps divert fluid into the first opening (118) and the pump loop (110). Protrusions (250) may extend from a wall of the transport channel (102). Protrusions (250) may be separate from the side walls of the transport channel (102). Protrusions (250) may span the entire channel from bottom to top or side to side, for example, like a column. Protrusions (250) may extend from a surface into the channel, like a stalactite or stalagmite. In an example, a single protrusion (250) is associated with each first opening (118). A first opening (118) may have multiple associated protrusions (250). Protrusions (250) may also impact the flow characteristics of fluid in the transport channel. Protrusions (250) may also be used to induce local turbulent flow for mixing. Protrusions (250) may be positioned to enhance pumping of the actuator (122), for example, by containing a bubble from impinging on the transport channel (102) or increasing back pressure.

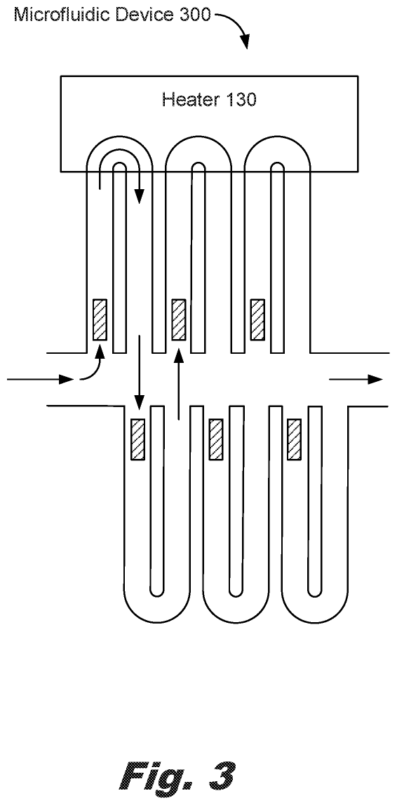

FIG. 3 shows an example of a microfluidic device (300) having pump loops (110) on both sides of the transport channel (102) consistent with this specification.

Allowing pump loops (110) on both sides of the transport channel (102) allows additional configurations to the microfluidic device (300). For example, one side can be used to support heating and the other cooling. The pump loops (110) may be organized into pairs of pump loops (110) and a first pump loop (110-1) and second pump loop (110-2) of a pair pump loops (110) are located on opposite sides of the transport channel (102). This may be advantageous when the second opening (120) of the first pump loop (110-1) of the pair of pump loops (110) is situated opposite the first opening (118) of the second pump loop (110-2) of the pair of pump loops (110) on either side of the transport channel (102). Aligning the second opening (120) of the first pump loop (110-1) which outputs fluid form the first pump loop (110-1) with the first opening (118) of the second pump loop (110-2) minimizes the resistance to flow of fluid between the pump loops (110). Unlike the design in FIG. 1, the fluid flows straight across the transport channel (102) and doesn't need to be redirected to move into the next pump loop (110) in the set of pump loops (110). This may improve the pumping efficiency of the actuators (122)

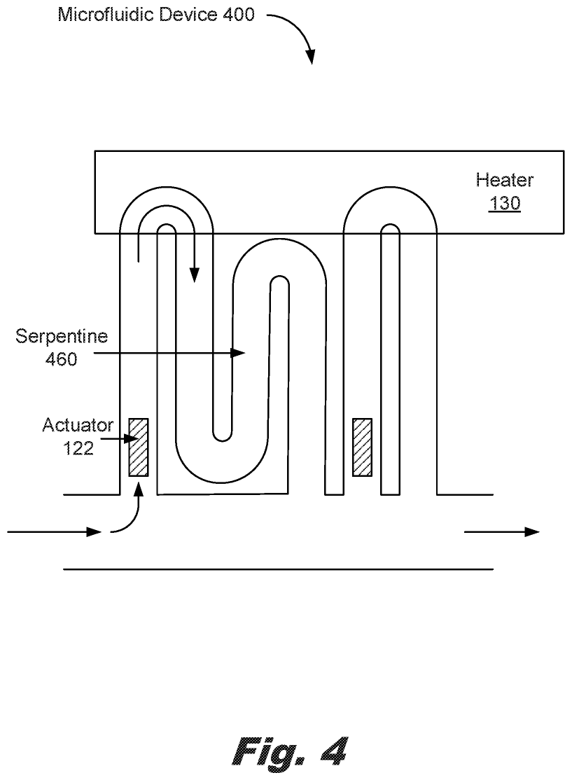

FIG. 4 shows an example of a microfluidic device with a pump loop having a serpentine section consistent with this specification. The serpentine (460) may be located between the first branch (112) and second branch (114) of the pump loop (110). The serpentine (460) may be part of the first branch (112). The serpentine (460) may be part of the second branch (460). The inclusion of a serpentine (460) provides additional length for the fluid to travel, for example, to allow more annealing time. In the configuration shown in FIG. 4 the serpentine (460) does not intersect with the heater (130). However, the serpentine (460) could be extended to contact the heating zone of the heater (130). Additional heaters (130), actuators (122), coolers, and/or other devices maybe added to the serpentine (460).

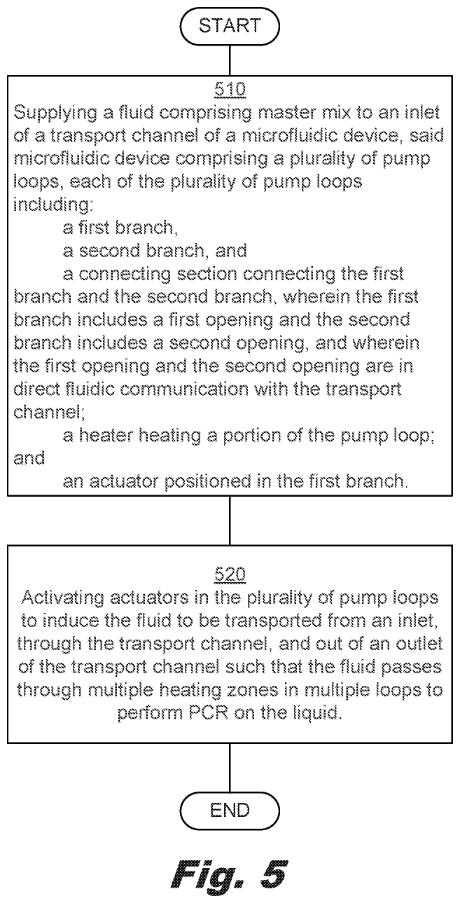

FIG. 5 shows a flowchart for a method (500) of performing Polymerase Chain Reaction (PCR) consistent with the present specification. The method (500) includes: supplying (510) a fluid comprising master mix to an inlet of a transport channel of a microfluidic device, said microfluidic device comprising a plurality of pump loops, each of the plurality of pump loops including: a first branch, a second branch, and a connecting section connecting the first branch and the second branch, wherein the first branch includes a first opening and the second branch includes a second opening, and wherein the first opening and the second opening are in direct fluid communication with the transport channel; a heater heating a portion of the pump loop; and an actuator positioned in the first branch; and activating (520) the actuators in the plurality of pump loops to induce the fluid to be transported from the inlet, through the transport channel, and to an outlet of the transport channel such that the fluid passes through multiple heating zones in multiple loops.

The method (500) is a method (500) of performing PCR. However, the claimed approach may be readily adapted to perform a variety of other processes where providing a range of temperatures over time is useful.

The method includes: supplying (510) a fluid comprising master mix to an inlet (104) of a transport channel (102) of a microfluidic device (100), said microfluidic device (100) including a plurality of pump loops (110), each of the plurality of pump loops (110) including: a first branch (112), a second branch (114), and a connecting section (116) connecting the first branch (112) and the second branch (114), wherein the first branch (112) includes a first opening (118) and the second branch (114) includes a second opening (120), and wherein the first opening (118) and the second opening (120) are in direct fluid communication with the transport channel (102); a heater (130) heating a portion of the pump loop (110); and an actuator (122) positioned in the first branch (112); and activating (520) the actuators (122) in the plurality of pump loops (110) to induce the fluid to be transported from the inlet (104), through the transport channel (102), and to an outlet (106) of the transport channel (102) such that the fluid passes through multiple heating zones in multiple pump loops (110).

The method (500) includes supplying (510) a fluid comprising master mix to an inlet (104) of a transport channel (102) of a microfluidic device (100), said microfluidic device (100) including a plurality of pump loops (110), each of the plurality of pump loops (110) including: a first branch (112), a second branch (114), and a connecting section (116) connecting the first branch (112) and the second branch (114), wherein the first branch (112) includes a first opening (118) and the second branch (114) includes a second opening (120), and wherein the first opening (118) and the second opening (120) are in direct fluid communication with the transport channel (102); a heater (130) heating a portion of the pump loop (110); and an actuator (122) positioned in the first branch (112). The fluid comprising master mix may contain a DNA sequence to be amplified. The fluid comprising master mix may be mixed prior to being provided to the inlet (104). Master mix is understood to include nucleotides and polymerase enzyme in a solvent, e.g., water. Master mix may include primers, buffers, salts, etc. Primers include both forward and reverse primers.

Supplying the fluid may include providing the fluid at an input (104) to the microfluidic device (100). In some examples, the fluid is provided to a port which is connected to the input (104). The fluid may be injected into a reservoir, where the reservoir supplies the input (104). The fluid may be mixed in the reservoir. The fluid may be premixed prior to providing the fluid to the microfluidic device (100). In some examples, the fluid is provided at a pressure greater than ambient. The fluid may be provided with a pressure head from its reservoir.

In an example, the microfluidic device (100) includes a sensor to detect when the fluid has been provided to the input (104) of the transport channel (102). This sensor may be a resistance or conductivity sensor which detects the presence of a liquid with ions bridging two electrical contacts. In an example, detecting the provision of fluid to the microfluidic device (100) provides a time point used for monitoring movement of the fluid through the heating zones in the pump loops (110).

The method (500) includes activating (520) the actuators (122) in the plurality of pump loops (110) to induce the fluid to be transported from the inlet (104), through the transport channel (102), and to an outlet (106) of the transport channel (102) such that the fluid passes through multiple heating zones in multiple pump loops (110). The fluid passes through multiple heated zones. The fluid passes through cooler zones between the heated zones. The temperature profile of heating and cooling are arranged to provide the thermal cycling supporting the two reactions of PCR, namely melting and copying. The multiple heated zones may be at different temperatures. The dwell time in different heated zones may vary from each other. In some examples, the temperature of the heated zone is modified depending on a calculated dwell time. In an example, the firing rate of the actuators (122) is adjusted based on a detected temperature in the heating zones.

The method (500) may further include measuring in a loop of the plurality of loops, a DNA concentration. In an example, the DNA concentration is measured using absorbance or based on binding.

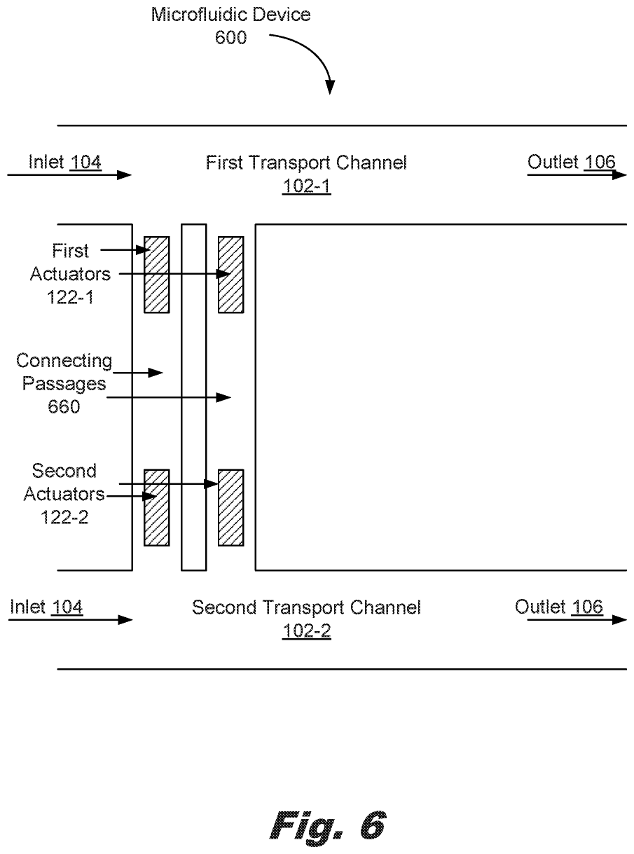

FIG. 6 shows a microfluidic device (600) with multiple transport channels (102) consistent with this specification. The microfluidic device (600) includes: a first and second transport channel (102), each having an inlet (104) and an outlet (106); a plurality of connecting passages (660) connecting the first and second transport channels (102), wherein each of the plurality of the connecting passages (660) includes: a first actuator (122-1) near the first transport channel (102-1); and a second actuator (122-2) near the second transport channel (102-2).

The microfluidic device (600) is a more generalized version of the device shown in FIG. 1. In contrast with the device of FIG. 1, the pump loop (110) is replaced with two connecting passages (660). The first connecting section(s) (116) have been combined to form a second transport channel (102). In order to return the fluid down the second connecting passage (660) an additional actuator (122) has been provided. Two other actuators (122) have been provided to allow reversing the flow in the connecting passages (660). Accordingly, the microfluidic device (600) includes more actuators and will there for have more control lines and similar overhead compared with the design of FIG. 1. However, the advantages of control provided by the additional actuators (122) are notable. As discussed, the ability to reverse the flow in the passages (660) is useful. The device of FIG. 6 may also be used to loop between the two passages (660) for any number of cycles desired. Further, the multiple inlets provide the ability to provide different mixes or different chemicals for different parts of the cycle.

The device of FIG. 6 may be augmented with heaters (130), chillers, measurement devices, etc. A sample can be provided at one of the inputs (104), looped through the connecting passages (760) a desired number of cycles, and then provided at an output (106) for further processing. Similarly, the disclosed design may be expanded, for example, by providing a third transport channel (102-3). Similar connecting passages (760) may be used to communicate with the third transport channel (102-3). Additional connecting passages (102) may be added as desired. The resulting structure allows materials to be processed, mixed, separated, processed more, etc. allowing a wide range of operations. Some of the connecting passages (760) may be equipped with sensors to monitor or evaluate processes or results. The resulting system can be used to provide a general set of “circuits” capable of performing a variety of reactions which may be combined or processed as needed. The flexibility of the approach described in FIG. 6 allows standardization of the microfluidic devices (600) while allowing adaptation of the device at time of use to the particular chemical process required. This approach reduces the cost of the microfluidic devices (600) by allowing them to be used for a wider variety of tasks and enjoying the associated economies of scale in production.

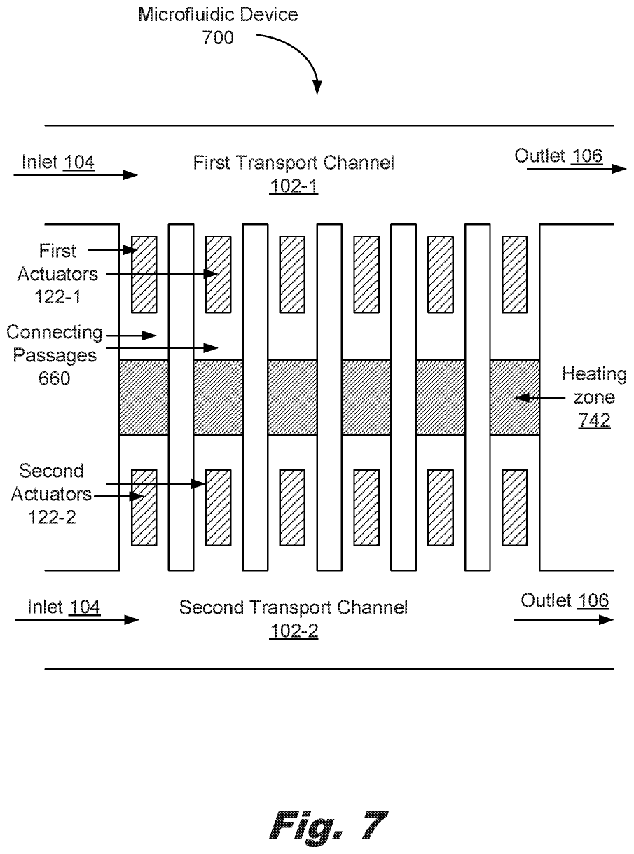

FIG. 7 shows a microfluidic device (700) with multiple transport channels (102) consistent with this specification. FIG. 7 builds on the design of FIG. 6 and adds additional connecting passages (760). FIG. 7 also adds heating zone (742). As discussed above with respect to the other microfluidic devices (200) heating zones (742) may be added as large zones. Heating zones (742) may be added with individual resistive heaters (132). Such individualized heaters (132) allow greater control over the temperature profile of the fluid in the microfluidic device (700), for example, allowing one connecting passage (760) to be warm and the adjacent connecting passage (760) to be cool.

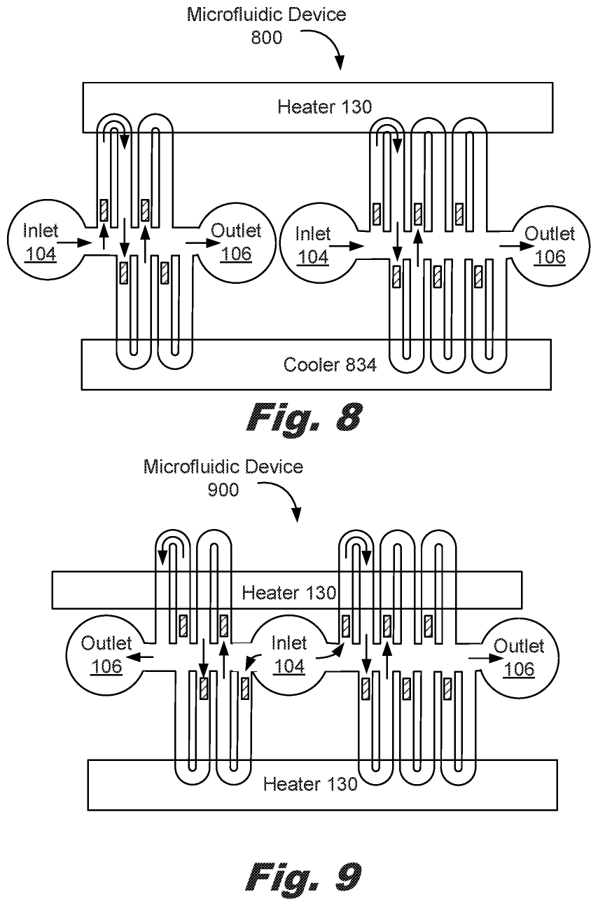

FIG. 8 shows an array of microfluidic devices (800) capable of parallel processing samples in an example consistent with this specification. In FIG. 8 multiple microfluidic devices (800) are arranged in a row. This allows a single heater (130) and/or cooler (834) to support multiple devices (800). Further, additional rows may be added above and below. This allows a large array of devices (800) which may be used to process many samples in parallel. The microfluidic device (800) may incorporate any of the variations described in this specification including combinations of elements from different explicit examples.

FIG. 9 shows a pair of microfluidic devices (900) capable of parallel processing samples in an example consistent with this specification. The devices of FIG. 9 share similarities with FIG. 8 and may be similarly expanded as described above. FIG. 9 also uses a single input (104) to supply multiple transport channels (102) and thereby feed multiple devices (900). Although only two devices (900) are shown here, expanding this concept to allow multiple of microfluidic devices to radiate from a common input (104) may facilitate replicates and/or other parallel processing. Although the two devices shown in FIG. 9 are similar, other combinations with different devices sharing a common input (104) may allow a large number of tests to be performed through a single input (104). The shared input (104) for example, may be connected to the output of an amplification section. The various microfluidic devices may then allocate different parts of the amplified sample and sequence different portions or using different processes, etc.

FIG. 9 also shares commonalities with FIG. 6 where multiple inputs (104) and multiple outputs (106) may be arranged together to form a network of microfluidic devices. The devices of FIGS. 6-9 may provide parallel processing of a large number of samples or replicates. In some examples, these are combined with an automated fluid ejector(s) and/or autodispenser to facilitate loading the inputs (104) of the different microfluidic devices (600, 700, 800, 900).

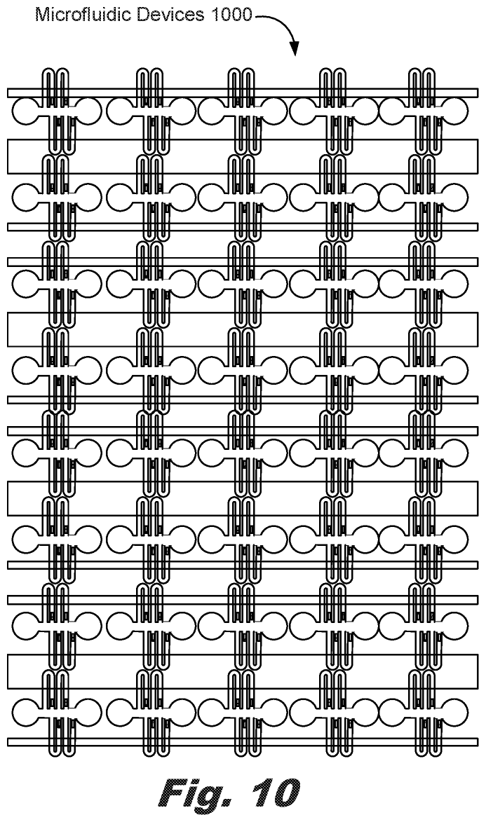

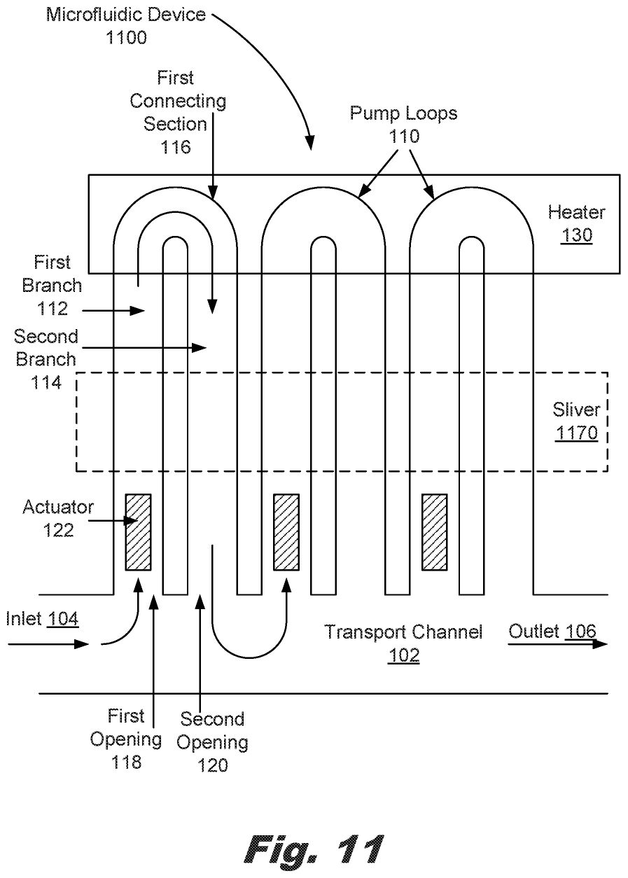

FIG. 10 shows an array of microfluidic devices (1000) according to an example consistent with this specification. In this example, the devices (1000) are all similar. However, in other examples the devices (1000) of the array may be heterogeneous. Similarly, while the inputs (104) and outputs (106) are shown as separate from each other, the inputs (104) and outputs (106) may be connected together to allow the microfluidic devices (1000) to feed adjacent devices (1000). This array of microfluidic devices (1000) may be particularly useful for digital PCR (dPCR) to assess DNA concentrations.

FIG. 11 shows an example of a microfluidic device (1100) consistent with the present specification. The device (1100) includes a silicon sliver (1170) molded into the substrate. The silicon silver(s) (1170) may be located at any point in the substrate and may contain electronic components, including, but not limited to: heaters (130), logics, thermal sinks, actuators (122), etc.

In one example the silicon sliver (1170) is narrow, for example on the order of 200 micrometers in width and depth. The silicon sliver (1170) may be overmolded or otherwise integrated into the microfluidic device (1100). The silicon sliver (1170) may be overmolded and then exposed by secondary processing. For example, the polymer body of the substrate, which forms the channels (102) and pump loops (110) may be molded or cast over the sliver (1170). A portion of the polymer may then be removed to expose part of the silver (1170), for example, electrical connections and/or other contact points. This approach allows efficient use of the silicon components while using the polymer to form mechanical features of the microfluidic device (1100).

It will be appreciated that, within the principles described by this specification, a vast number of variations exist. It should also be appreciated that the examples described are only examples, and are not intended to limit the scope, applicability, or construction of the claims in any way.

What is claimed is:

1. A microfluidic device comprising:
a transport channel having an inlet and an outlet;
a plurality of pump loops extending along the transport channel, wherein each of the plurality of pump loops comprises:
a first branch, a second branch, and a first connecting section connecting the first branch and the second branch,
wherein the first branch includes a first opening and the second branch includes a second opening, and
wherein the first opening and the second opening are in direct fluid communication with the transport channel;
an actuator positioned in the first branch; and
a heater positioned to heat fluid in a portion of the pump loop.

2. The device of claim 1, wherein the heater provides heating to multiple adjacent pump loops.

3. The device of claim 1, wherein each pump loop comprises an individual resistive heater such that adjacent pump loops may be heated to different temperatures.

4. The device of claim 1, wherein each of the plurality of pump loops further comprises a cooling zone.

5. The device of claim 1, wherein each of the plurality of pump loops further comprises a second heater to apply heating to a separate portion of the pump loop.

6. The device of claim 1, further comprising a plurality of protrusions positioned in the transport channel to facilitate fluid flow from the second opening of one pump loop to the first opening of an adjacent pump loop.

7. The device of claim 1, wherein the plurality of pump loops are organized into pairs of pump loops and a first pump loop and second pump loop of a pair pump loops are located on opposite sides of the transport channel.

8. The device of claim 7, wherein the second opening of the first pump loop of the pair of pump loops is situated opposite the first opening of the second pump loop of the pair of pump loops in the transport channel.

9. The device of claim 1, wherein a pump loop of the plurality of pump loops further comprises a second actuator in the second branch of the pump loop.

10. The device of claim 1, wherein a branch of a pump loop comprises a serpentine section.

11. A method of performing Polymerase Chain Reaction (PCR) comprising:
supplying a fluid comprising master mix to an inlet of a transport channel of a microfluidic device, said microfluidic device comprising a plurality of pump loops, each of the plurality of pump loops comprising:
a first branch,
a second branch, and
a connecting section connecting the first branch and the second branch, wherein the first branch includes a first opening and the second branch includes a second opening, and wherein the first opening and the second opening are in direct fluid communication with the transport channel;
a heater heating a portion of the pump loop; and
an actuator positioned in the first branch; and
activating the actuators in the plurality of pump loops to induce the fluid to be transported from the inlet, through the transport channel, and to an outlet of the transport channel such that the fluid passes through multiple heating zones in multiple pump loops.

12. The method of claim 11, further comprising measuring in a loop of the plurality of loops, a DNA concentration.

13. The method of claim 12, further comprising measuring in multiple loops of the plurality of loops, a DNA concentration.

14. A microfluidic device comprising:
a first and second transport channel, each having an inlet and an outlet;
a plurality of connecting passages connecting the first and second transport channels, wherein each of the plurality of the connecting channels comprises:
a first actuator near the first transport channel; and
a second actuator near the second transport channel.

15. The system of claim 14, wherein each transport channel further comprises a heating zone.
